(19) United States
(12) Patent Application Publication
(10) Pub. No.: US 2020/0035352 A1
(43) Pub. Date: Jan. 30, 2020

(54) SERVER, CONTROL SYSTEM, CONTROL METHOD AND NON-TRANSITORY COMPUTER READABLE MEDIUM

(71) Applicant: HITACHI HIGH-TECHNOLOGIES CORPORATION, Tokyo (JP)

(72) Inventors: Nobuhiro SEKIMOTO, Tokyo (JP); Masataka OKAYAMA, Tokyo (JP); Juntaro ARIMA, Tokyo (JP); Yoshihisa MATSUI, Tokyo (JP)

(21) Appl. No.: 16/335,709

(22) PCT Filed: Sep. 21, 2017
(86) PCT No.: PCT/JP2017/034137

§ 371 Date: Mar. 22, 2019

(30) Foreign Application Priority Data
Sep. 23, 2016 (JP) 2016-186222

(51) Int. Cl. G16H 40/20

(52) U.S. Cl. CPC G16H 40/20; H04L 67/12

(57) ABSTRACT

The present invention controls a device that constitutes the cause of disturbance and improves accuracy during the use of a prescribed device. A server provided with: a storage unit for storing reservation information for using a first device that includes information corresponding to the start date/time and end date/time of use of the first device; and a processing unit for transmitting, to a control terminal that controls a second device, a first indication for placing the second device into a prescribed state when the start date/time based on the reservation information stored in the storage unit is reached, and transmitting, to the control terminal, a second indication for placing the second device into a state prior to the start date/time when the end date/time based on the reservation information stored in the storage unit is reached.

### TECHNICAL FIELD

The present invention pertains to a server, a control system, a control method and a control program.

### BACKGROUND ART

Measurement/medical/industrial apparatuses utilizing irradiations and behaviors of charged particles are instanced by electron microscopic apparatuses and ion beam radiotherapy apparatuses. In these apparatuses, when the behaviors of the charged particles fluctuate due to disturbances, there increase possibilities of causing a scatter in measurement value, blurs or blurring of images formed from an intensity distribution and a decrease in therapy effect. Herein, the disturbances are exemplified by apparatus fluctuations caused by vibrations, sounds, wind and other equivalent elements, variations in electromagnetic wave, electric field and magnetic field, fluctuations in voltage and electric current of a power source, and variations in radiation quantity. Factors of the disturbances are instanced by the wind from an air conditioner, the electromagnetic waves from an microwave oven, radios and other equivalent devices, the vibrations from the apparatuses having a motor and other equivalent devices, and operations of apparatuses consuming a large amount of electric power.

### DOCUMENTS OF PRIOR ARTS

### Patent Documents

[Patent Document 1] Japanese Patent Application Laid-Open Publication No. 2011-158186

[Patent Document 2] Japanese Patent Publication No. 5452160.

### SUMMARY

### Problems to be solved by the Invention

The electron microscopic apparatus and the ion beam radiotherapy apparatus are required to control ambient environments disturbing the behaviors of the particles (to eliminate factors of disturbances) in order to improve accuracy in acquiring measurement values and the images of the intensity distribution and in converging positions of particle radiating regions. It is, however, quite difficult to control an equipment becoming the factor of disturbance in order to improve the accuracy of the apparatus instanced by the electron microscopic apparatus.

The present invention aims at improving accuracy when using a predetermined apparatus by controlling an equipment becoming a factor of disturbance.

### Means for Solving the Problems

A solution of the problems described above involves adopting the following means.

To be specific, according to a first aspect, a server includes: a storage unit to store reservation information for using a first apparatus, the reservation information containing items of information corresponding to a starting day and time and a finishing day and time for using the first apparatus; and a processing unit to transmit a first instruction for setting a second apparatus in a predetermined status to a control terminal to control the second apparatus when reaching the starting day and time based on the reservation information stored in the storage unit, and to transmit a second instruction for setting the second apparatus in a status before the starting day and time to the control terminal when reaching the finishing day and time based on the reservation information stored in the storage unit.

The aspect of the disclosure may be accomplished in such a way that an information processing apparatus runs a program. In other words, the configuration of the disclosure may be specified as a program for making the information processing apparatus execute processes to be executed by respective means in the aspect described above, or as a non-transitory computer readable recording medium on which the program is recorded. The configuration of the disclosure may also be specified as a method by which the information processing apparatus executes the processes to be executed by the respective means. The configuration of the disclosure may further be specified as a system including the information processing apparatus that executes the processes to be executed by the respective means.

Steps of writing the program include, of course, processes to be executed in time-series along a described sequence, and processes to be executed in parallel or individually without necessarily being executed in time-series. A part of steps of writing the program may also be omitted.

### Effect of the Invention

According to the present invention, it is feasible to improve the accuracy when using the predetermined apparatus by controlling the equipment becoming the factor of disturbance.

### BRIEF DESCRIPTION OF THE DRAWINGS

FIG. 1 is a diagram illustrating an example of a configuration of a system according to an embodiment.

FIG. 2 is a diagram illustrating an example of a table stored in a storage unit 104 of a server 100.

FIG. 3 is a diagram illustrating an example of a hardware configuration of an information processing apparatus.

FIG. 4 is a flowchart illustrating an operation flow of the server according to the embodiment.

FIG. 5 is a chart illustrating an example of a schedule table.

FIG. 6 is a chart illustrating an example of a reserved status screen displayed on a display unit of a reservation terminal.

FIG. 7 is a chart illustrating an example of the reserved status screen displayed on the display unit of the reservation terminal.

FIG. 8 is a chart illustrating an example of an apparatus associative table.

FIG. 9 is a chart illustrating an example of an apparatus table.

FIG. 10 is a chart illustrating an example of an apparatus control table.

### EMBODIMENTS

### Mode for Carrying out the Invention

An embodiment will hereinafter be described with reference to the drawings. A configuration of the embodiment is an exemplification, and a configuration of the present invention is not limited to the specific configuration of the embodiment of disclosure. A specific configuration corresponding to the embodiment may be properly adopted when carrying out the invention.

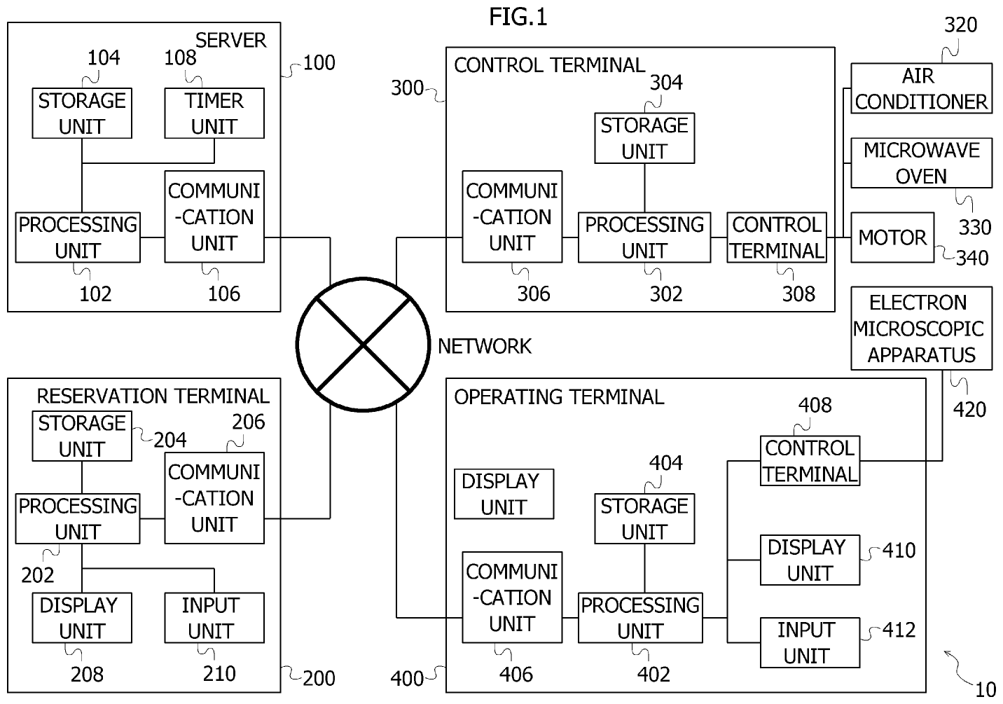

FIG. 1 is a diagram illustrating a configuration of a system according to the embodiment. A system 10 according to the embodiment includes a server 100, a reservation terminal 200, a control terminal 300, and an operating terminal 400. The server 100, the reservation terminal 200, the control terminal 300 and the operating terminal 400 are connected to each other via a network. An air conditioner 320, a microwave oven 330 and a motor 340 are connected to the control terminal 300. An electron microscopic apparatus 420 is connected to the operating terminal 400. Herein, the air conditioner 320, the microwave oven 330 and the motor 340, which are equipments having factors of causing disturbances, are controlled for improving accuracy when using the electron microscopic apparatus 420. The equipments having the factors of causing the disturbances are not limited to those instanced herein. Apparatuses with accuracies to be improved, are not limited to the electron microscopic apparatus 420. Apparatuses with accuracies to be improved, e.g., the apparatuses using accelerated electron beams, proton beams and particle beams are typically exemplified by, i.e., a Transmission Electron Microscope (which will hereinafter be abbreviated to TEM), a Scanning Electron Microscope (which will hereinafter be abbreviated to SEM), a radiation therapy apparatus and an ion beam radiotherapy apparatus. The TEM, the SEM and the ion beam radiotherapy apparatus have such potentialities that the apparatuses themselves become equipments having the factors of causing the disturbances against other apparatuses with the accuracies to be improved. The system 10 may include a plurality of apparatuses with the the accuracies to be improved. The system 10 may include, though one control terminal 300 is illustrated herein, a plurality of control terminals 300 that control the equipments having the factors of causing the disturbances. Such a configuration may be available as to implement a function of controlling the equipments in the same way as the control terminal 300 does on another operating terminal 400 to which another electron microscopic apparatus 420 and other equivalent apparatuses having the factors of causing the disturbances are connected.

The server 100 includes a processing unit 102, a storage unit 104, a communication unit 106, and a timer unit 108. The processing unit 102 gives an instruction of controlling the equipments having the factors of causing the disturbances, based on a usage reservation schedule of the electron microscopic apparatus 420 and other equivalent apparatuses. The storage unit 104 stores data, tables, programs and other equivalent software that are used in the server 100. The communication unit 106 transmits and receives the data and signals to and from (performs communications with) other apparatuses via the network. The timer unit 108 counts time.

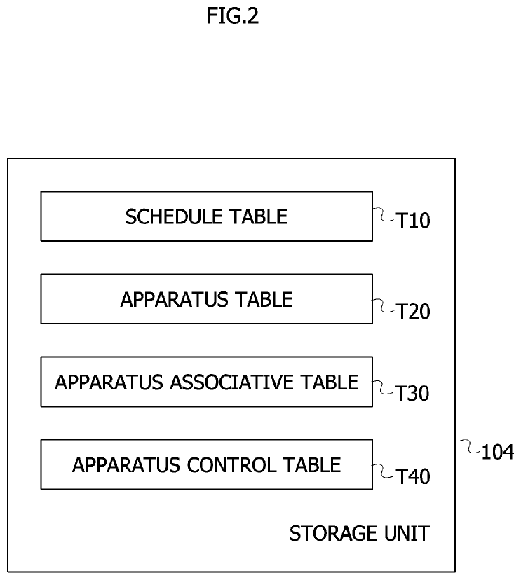

FIG. 2 is a diagram illustrating an example of tables that are stored in the storage unit 104 of the server 100. The storage unit 104 stores a schedule table T10, an apparatus table T20, an apparatus associative table T30, and an apparatus control table T40. Contents of the respective tables will be described later.

The reservation terminal 200 includes a processing unit 202, a storage unit 204, a communication unit 206, a display unit 208, and an input unit 210. The processing unit 202 executes a reservation accepting process for using the electron microscopic apparatus 420 and other equivalent apparatuses. The storage unit 204 stores the data, the tables, the programs and other equivalent software that are used in the reservation terminal 200. The communication unit 206 transmits and receives the data and the signals to and from (performs the communications with) other apparatuses via the network. The display unit 208 displays, to a user, a reserved status screen and a reservation accepting screen of the electron microscopic apparatus 420 and other equivalent apparatuses. The input unit 210 accepts a reservation for using the electron microscopic apparatus 420 and other equivalent apparatuses from the user.

The control terminal 300 includes a processing unit 302, a storage unit 304, a communication unit 306, and a control unit 308. The processing unit 302 controls an operation of the equipment having the factor of causing the disturbance upon receiving an instruction from the server 100. The storage unit 304 stores the data, the tables, the programs and other equivalent software that are used in the control terminal 300. The communication unit 306 transmits and receives the data and the signals to and from (performs the communications with) other apparatuses via the network. The control unit 308 controls, based on an instruction given from the processing unit 302, the operations of the equipments connected thereto. The control unit 308 acquires operating statuses of the equipments connected thereto. The air conditioner 320, the microwave oven 330 and the motor 340 are connected to the control unit 308.

The operating terminal 400 includes a processing unit 402, a storage unit 404, a communication unit 406, a control unit 408, a display unit 410, and an input unit 412. The processing unit 402 instructs the control unit 408 to control the operation of the electron microscopic apparatus 420. The storage unit 404 stores the data, the tables, the programs and other equivalent software that are used in the operating terminal 400. The communication unit 406 transmits and receives the data and the signals to and from (performs the communications with) other apparatuses via the network. The control unit 408 controls, based on an instruction given from the processing unit 402, the operations of the apparatuses connected thereto. The display unit 410 is an output means that displays a result and other equivalent items with respect to the electron microscopic apparatus 420. The input unit 412 is an input means for operating the electron microscopic apparatus 420.

Each of the server 100, the reservation terminal 200, the control terminal 300 and the operating terminal 400 is attainable by using a dedicated or general-purpose computer instanced by a PC (Personal Computer), a smartphone, a mobile phone, a tablet terminal, a car navigation system, a PDA (Personal Digital Assistant) and a workstation (WS) or by using an electronic equipment mounted with the computer.

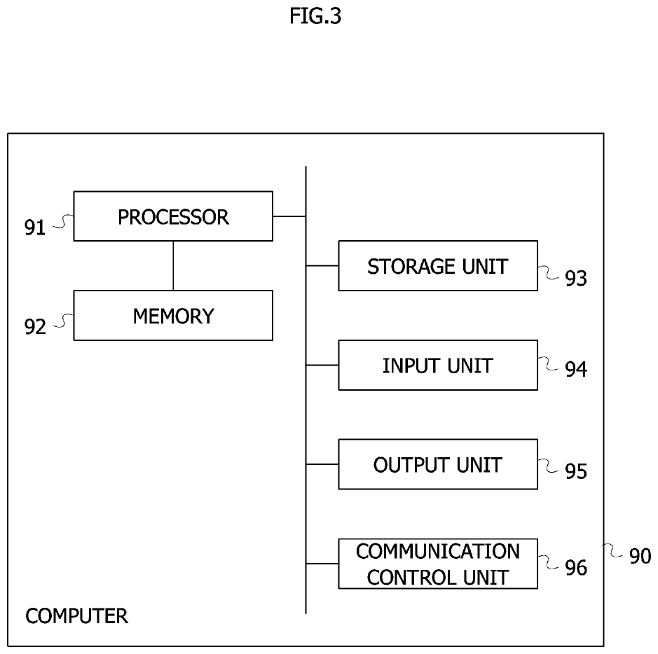

FIG. 3 is a diagram illustrating a hardware configuration of an information processing apparatus. The information processing apparatus depicted in FIG. 3 has a configuration of the general computer. Each of the server 100, the reservation terminal 200, the control terminal 300 and the operating terminal 400 is attained by a computer 90 as illustrated in FIG. 3. The computer 90 in FIG. 3 includes a processor 91, a memory 92, a storage unit 93, an input unit 94, an output unit 95, and a communication control unit 96. The memory 92 is connected directly to the processor 91, and the components other than the memory 92 are interconnected via a bus. The storage unit 93 is a non-transitory computer readable recording medium. The hardware configuration of the computer may, without being limited to the example illustrated in FIG. 3, properly omit, replace and add the components.

The processor 91 loads the programs, the various items of data and the various types of tables stored on the recording medium into a working area of the memory 92 and then executes these software components, and the respective constructive units are thereby controlled through running the programs, whereby the computer 90 is enabled to attain functions matching with predetermined purposes.

The processor 91 is instanced by a CPU (Central Processing Unit) and a DSP (Digital Signal Processor).

The memory 92 includes, e.g., a RAM (Random Access Memory) and a ROM (Read Only Memory). The memory 92 is also called a main storage device.

The storage unit 93 is, e.g., an EPROM (Erasable Programmable ROM) and a Hard Disk Drive (HDD). The storage unit 93 may include a removable medium, i.e., a portable recording medium. The removable medium is a disc recording medium instanced by a USB (Universal Serial Bus) memory, or a CD (Compact Disc) and a DVD (Digital Versatile Disc). The storage unit 93 is also called a secondary storage device.

The storage unit 93 stores the various categories of programs, the various items of data and the various types of tables on the recording medium in a readable/writable manner. The storage unit 93 stores an Operating System (OS), the various categories of programs, the various types of tables and other equivalent software components. The information to be stored in the storage unit 93 may also be stored in the memory 92. The information to be stored in the memory 92 may also be stored in the storage unit 93.

The OS is software operating as an intermediary between the software and the hardware, and for managing a memory space, files, processes and tasks. The OS includes a communication interface. The communication interface is a program for transmitting and receiving the data to and from other external devices connected via the communication control unit 96. The external devices embrace, e.g., other computers, external storage devices and other equivalent devices.

The input unit 94 includes a keyboard, a pointing device, a wireless remote controller, a touch panel and other equivalent devices. The input unit 94 may also include a video/image input device instanced by a camera, and a voice/sound input device instanced by a microphone.

The output unit 95 includes a display device instanced by an LCD (Liquid Crystal Display), an EL Electroluminescence) panel, a CRT (Cathode Ray Tube) display, and a PDP (Plasma Display Panel), and an output device instanced by a printer. The output unit 95 may also include a voice/sound output device instanced by a loudspeaker.

The communication control unit 96 establishes connections to other apparatuses and controls communications between the computer 90 and other apparatuses. The communication control unit 96 is instanced by a LAN (Local Area Network) interface board, a wireless communication circuit for wireless communications, and a communication circuit for wired communications. The LAN interface board and the wireless communication circuit are connected to a network exemplified by the Internet.

Processors load programs stored in auxiliary storage devices into the main storage devices and run the programs, whereby computers, which attain the server 100, the reservation terminal 200, the control terminal 300 and the operating terminal 400, implement functions as respective functional units. On the other hand, respective storage units of the server 100, the reservation terminal 200, the control terminal 300 and the operating terminal 400 are provided in storage areas of the main storage devices or the auxiliary storage devices.

### Operational Example

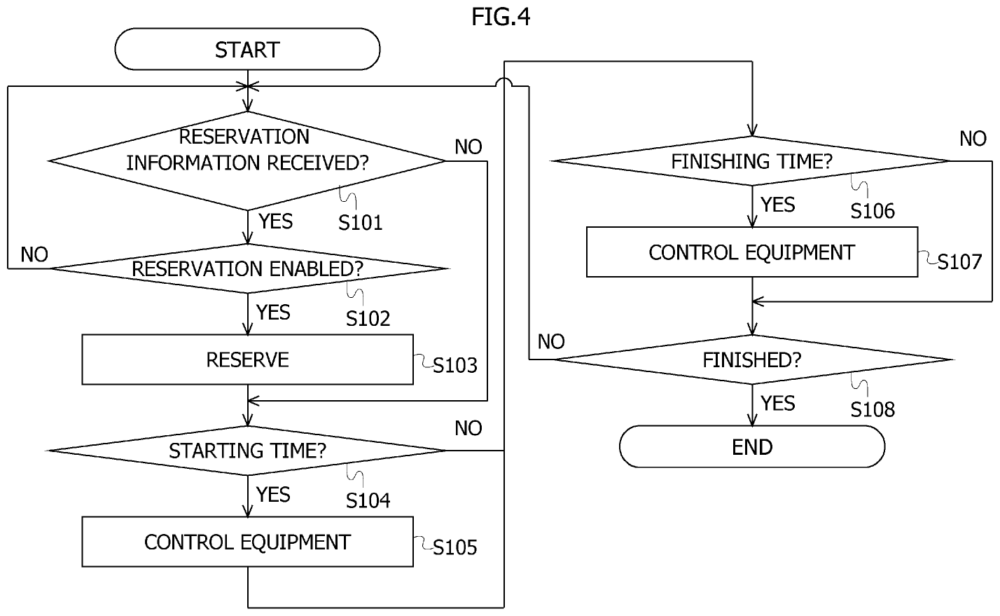

FIG. 4 is a flowchart illustrating an example of an operation flow of the server according to the embodiment. The reservation terminal 200, the control terminal 300 and the operating terminal 400 are connected to the server 100 in a communicable manner via the network.

The user, who tries using the electron microscopic apparatus 420, makes a reservation for using the electron microscopic apparatus 420 by employing the reservation terminal 200. Upon reaching a reserved day and time (starting day and time) of the electron microscopic apparatus 420, the user operates the electron microscopic apparatus 420 via the operating terminal 400. Hereat, operations of the equipments hindering the improvement of accuracy in the use of the electron microscopic apparatus 420 are controlled based on an instruction given to the control terminal 300 from the server 100.

A start of the operation flow in FIG. 4 is triggered by the user's instructing the reservation terminal 200 to start reserving the use of the electron microscopic apparatus 420 (assumed to be the SEM). The instruction of the start is made by, e.g., the user's starting up a predetermined application for the reservation on the reservation terminal 200. The reservation terminal 200 requests the server 100 for schedule information of the SEM to be reserved by the user through the application described above.

In S101, the processing unit 102 of the server 100 extracts a schedule table T10 stored in the storage unit 104. The processing unit 102 extracts, from the schedule table T10, reserved statuses of the respective apparatuses including the SEM to be reserved by the user.

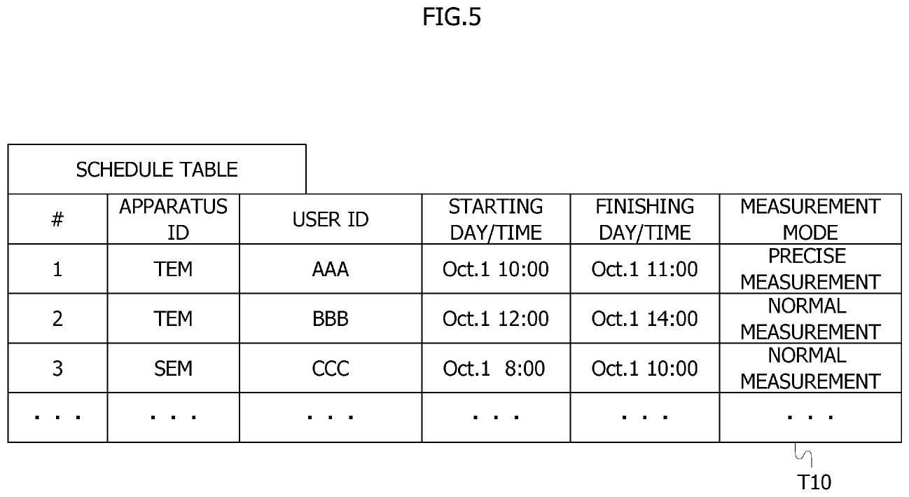

FIG. 5 is a chart illustrating an example of the schedule table. Reservations for using the electron microscopic apparatus 420 and other equivalent apparatuses are stored in the schedule table T10. An apparatus ID for the electron microscopic apparatus 420 and other equivalent apparatuses to be used, a user ID for identifying the user making the reservation, a usage starting day and time, a usage finishing day and time and a measurement mode for the use are associated with each other as reserved statuses in the schedule table T10. Herein, the measurement mode is classified into a precise measurement mode and a normal measurement mode, in which a time zone of a certain apparatus reserved for use in, e.g., the precise measurement mode inhibits other apparatuses from being reserved for use. This is because the operations of other apparatuses have apprehension of affecting the measurement of the apparatus with the measurement being made in the precise measurement mode.

The processing unit 102 transmits the extracted reserved statuses to the reservation terminal 200 via the communication unit 106. The processing unit 202 of the reservation terminal 200 receives the reserved statuses of the electron microscopic apparatus 420 and other equivalent apparatuses via the communication unit 206, and stores the reserved statuses in the storage unit 204. The processing unit 202 displays the received reserved statuses on the display unit 208.

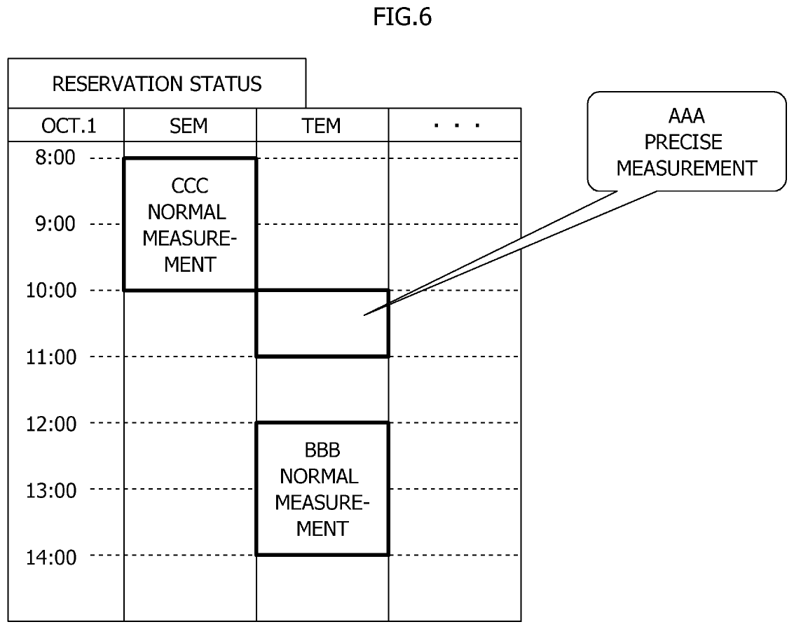

FIG. 6 is a chart illustrating an example of a reserved status screen to be displayed on the display unit of the reservation terminal. It is understood from the reserved status screen in FIG. 6 that “normal measurement” made by a user “CCC” is reserved for a period from 8:00 to 10:00 on October 1 with respect to the SEM, while with respect to the TEM, “precise measurement” made by a user “AAA” is reserved for a period from 10:00 to 11:00 on October 1, and “normal measurement” made by a user “BBB” is reserved for a period from 12:00 to 14:00 on October 1. The user, who tries using the electron microscopic apparatus 420, checks the reserved status screen displayed on the display unit 208 and is thereby enabled to examine the apparatus and the time zone (the starting day and time, the finishing day and time) to be reserved for the use by the user himself or herself.

The processing unit 202 of the reservation terminal 200 displays a reservation accepting screen on the display unit 208. The reservation accepting screen is provided with input fields of the apparatus to be reserved for the use by the user, the time zone and the measurement mode. The user inputs the apparatus ID for identifying the apparatus to be reserved for the use by the user, the time zone, the measurement mode and a user ID for identifying the user through the input unit 210 of the reservation terminal 200.

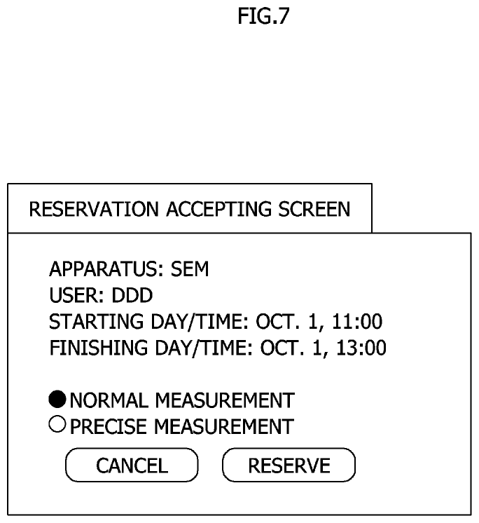

FIG. 7 is a chart illustrating an example of the reservation accepting screen displayed on the display unit of the reservation terminal. The reservation accepting screen in FIG. 7 is provided with fields for inputting the apparatus ID for identifying the apparatus desired for the user, the user ID, the starting day and time, the finishing day and time and the measurement mode (the normal measurement, or the precise measurement). The reservation accepting screen may be displayed together with the reserved status screen. Herein, the time zone for using the apparatus is specified by the starting day and time and the finishing day and time, and may also be specified by the starting day and time and a using period (time).

The processing unit 202 of the reservation terminal 200, upon inputting the items of reservation information (the apparatus, the user ID, the starting day and time, the finishing day and time, and the measurement mode) via the input unit 210, stores the reservation information in the storage unit 204. The processing unit 202 transmits the inputted reservation information to the server 100 via the communication unit 206.

The processing unit 102 of the server 100 determines whether the reservation information is received from the reservation terminal 200. When the reservation information is not received (S101; NO), the processing proceeds to S104. Whereas when the reservation information is received (S101; YES), the processing unit 102 stores the received reservation information in the storage unit 104. Thereafter, the processing diverts to S102.

In S102, the processing unit 102 determines whether the reservation based on the received reservation information is acceptable. The processing unit 102 compares the apparatus and the time zone (from the starting day and time to the finishing day and time) of the received reservation information with the items of reservation information of the schedule table T10 stored in the storage unit 104. When the reservation information of the schedule table T10 is overlapped with the received reservation information with respect to the same apparatus, the processing unit 102 determines that the reservation is unacceptable. The measurement mode of the received reservation information is the precise measurement mode, and the schedule table T10 contains the reservation information of a different apparatus in the time zone contained in the received reservation information, in which case the processing unit 102 determines that the reservation is unacceptable when an affecting degree is intermediate or large by taking the affecting degree between the apparatuses into consideration. The processing unit 102 may determine that the reservation is acceptable in cases other than the above-mentioned, e.g., when there is no overlapped reservation, when the measurement modes thereof are both the normal measurement modes, alternatively when not affecting in consideration of the affecting degree between the apparatuses or when the affecting degree is small.

When determining that the reservation is unacceptable (S102; NO), the processing unit 102 transmits information purporting that the reservation is unacceptable to the reservation terminal 200 via the communication unit 106. The processing unit 202 of the reservation terminal 200, upon receiving the information purporting that the reservation is unacceptable via the communication unit 206, displays the purport of the reservation being unacceptable on the display unit 208. The user having checked the display recognizes that the reservation is unacceptable, and may input a new reservation on the reservation accepting screen. On the other hand, the processing in the server 100 loops back to S101.

When determining that the reservation is acceptable (S102; YES) the processing unit 102 transmits information purporting that the reservation is acceptable to the reservation terminal 200 via the communication unit 106. The processing unit 202 of the reservation terminal 200 upon receiving the information purporting that the reservation is acceptable via the communication unit 206 displays the purport of the reservation being acceptable on the display unit 208. The user having checked the display recognizes that the reservation has been made. On the other hand, the processing in the server 100 proceeds to S103.

In S103, the processing unit 102 registers the received reservation information in the schedule table T10. The processing unit 102 stores the schedule table T10 registered with the received reservation information in the storage unit 104.

In S104, the processing unit 102 acquires information of the present day and time from the timer unit 108. The processing unit 102 determines whether the present day and time is coincident with the starting day and time of the reservation information contained in the schedule table T10 stored in the storage unit 104. When coincident with each other (S104; YES), the processing proceeds to S105. Whereas when not coincident (S104; NO), the processing diverts to S106.

In S105, the processing unit 102 of the server 100 extracts the apparatus ID of the reservation information, which reaches the starting day and time in S104. The apparatus (the electron microscopic apparatus or another equivalent apparatus) associated with the apparatus ID is the apparatus to be used by the user. The processing unit 102 extracts a record associated with the extracted apparatus ID from the apparatus associative table T30 stored in the storage unit 104. The apparatus associative table T30 is a table illustrating how the apparatuses to be used are associated with factors of disturbances on the occasion of using the apparatuses. The apparatus associative table T30 is a table that stores an apparatus (target apparatus) possibly becoming the factor of disturbance, an event (target event) possibly becoming the factor of disturbance, and an affecting degree with respect to the disturbance apparatus on the occasion of using the apparatus associated with the apparatus ID in a way that associates these items with the apparatus ID.

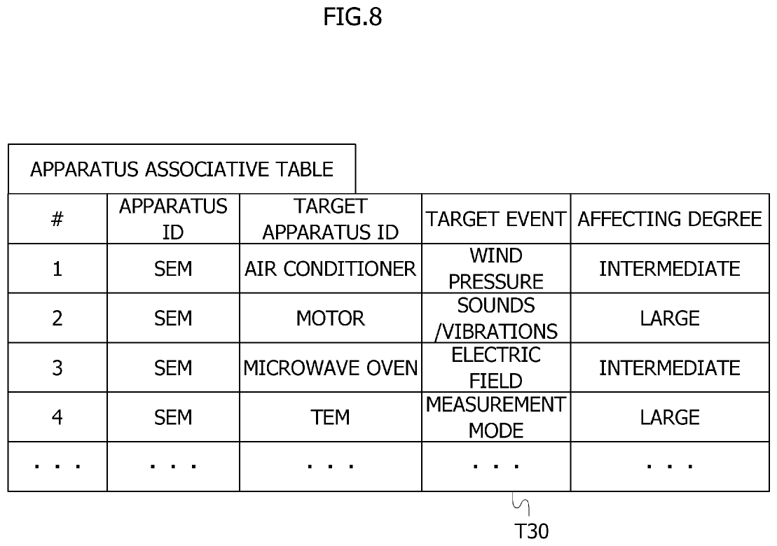

FIG. 8 is a chart illustrating an example of the apparatus associative table. The apparatus associative table T30 in FIG. 8 stores the apparatus ID associated with the apparatus to be used, the target apparatus ID associated with the equipment becoming the factor of disturbance with respect to the apparatus concerned, the target event (vibrations, an electric field and other equivalent events) possibly becoming the factor of disturbance, and the affecting degree with respect to the disturbance apparatus by being associated with each other. The affecting degree is herein expressed by “Large”, “Intermediate” and “Small”, and may also be expressed by a numerical value becoming larger as the affecting degree rises. The equipments becoming the factors of disturbances may include other electron microscopic apparatuses, ion beam radiotherapy apparatuses and other equivalent apparatuses.

The processing unit 102 extracts, from the apparatus table T20 stored in the storage unit 104, a record associated with the target apparatus ID contained in the records extracted from the apparatus associative table T30. The apparatus table T20 is a table illustrating how the apparatus is associated with a model number, a setting position, a control method and other equivalent items.

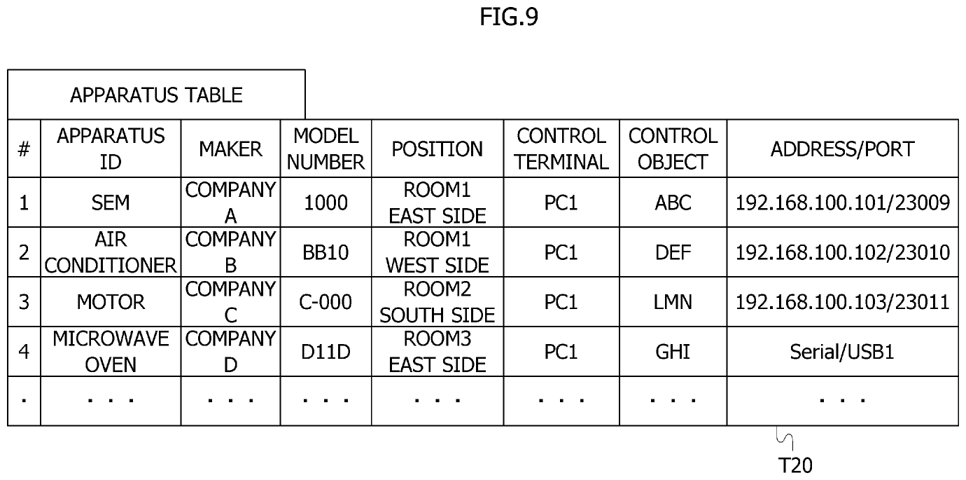

FIG. 9 is a chart illustrating an example of the apparatus table. The apparatus table T20 in FIG. 9 stores the apparatus ID associated with the apparatus, a maker manufacturing the apparatus concerned, the model number of the apparatus, the setting position of the apparatus, a control terminal controlling the apparatus, a control object representing a program for controlling the apparatus and specifications, and an address/port indicating an access route to the apparatus from the control terminal by being associated with each other. The control terminal is connected to the apparatus via the network, in which case an address on the network is registered in an “address/port” field. The control terminal is connected directly to the apparatus, in which case a connection port is registered in the “address/port” field. The connection between the control terminal 300 and the apparatus may be established through communications using a USB, a serial connection, a LAN, infrared-rays and other equivalent means. Note that the “address/port” field may be URI (Uniform Resource Identifier)—formatted for uniquely identifying an interface for controlling resources of the controlled apparatus. When the controlled apparatus opens an API (Application Program Interface) for the control to the public by using a Web technology, a control service thereof may also be utilized.

The control object for controlling an air-conditioning equipment instanced by an air conditioner is exemplified by H-LINK/H-LINK2 and DIII-NET. The control object enables a temperature, an airflow rate and other equivalent elements to be controlled. The control object for controlling illumination, electric power, air conditioning and other equivalent elements in a building and other equivalent structures is exemplified by LONWORKS, BAC-net, X10, KNX (Konnex), JEM-A, and ECHONET-LITE. The control object for controlling home electrical appliances is exemplified by UPnP, DLNA (registered trademark), and IGRS. The control object is selected properly corresponding to the equipment to be controlled. Other existing technologies enable the control over the equipments possibly becoming the factors of disturbances.

The processing unit 102 extracts, from the apparatus control table T40 stored in the storage unit 104, a record exhibiting coincidences of the target apparatus ID, the target event and the affecting degree that are contained in the record extracted from the apparatus associative table T30. The apparatus control table T40 is a table in which to store control contents corresponding to the target apparatus, the target event and the affecting degree.

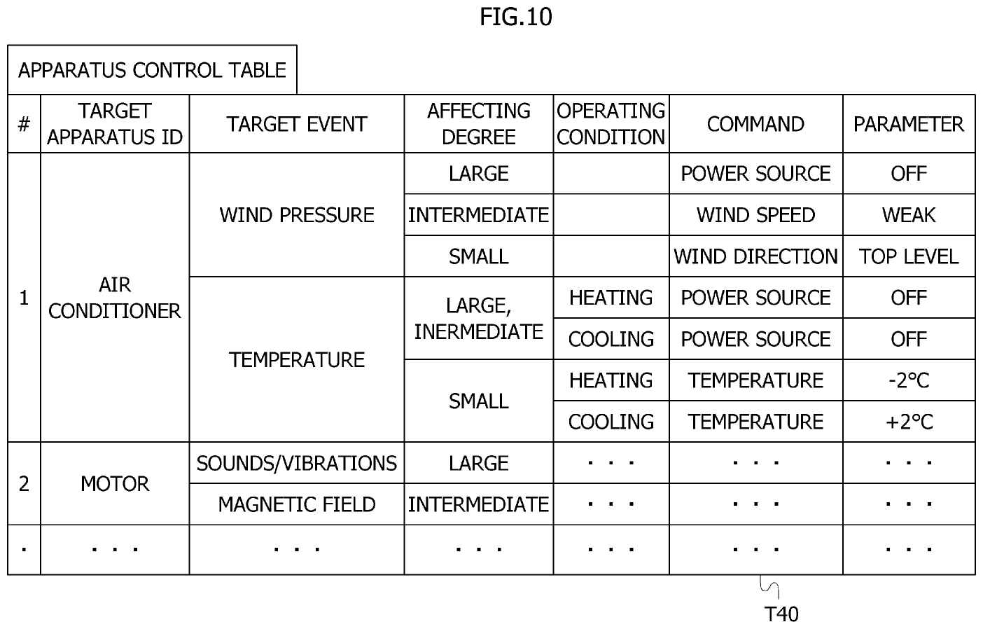

FIG. 10 is a chart illustrating an example of the apparatus control table. The apparatus control table T40 in FIG. 10 stores the target apparatus ID of the control target apparatus, the target event possibly becoming the factor of disturbance, the affecting degree of the disturbance, a control command, an operating condition of the command, and a parameter for the command by being associated with each other. For example, the target apparatus ID is “air conditioner”, the target event is “temperature”, and the affecting degree is “large”, in which case the parameter is set to “−20° C.” by the control command “temperature” when an operation mode of the air conditioner is “heating”. The temperature of the air condition is thereby decreased by 2° C.

The processing unit 102 transmits an instruction of controlling the apparatus becoming the factor of disturbance via the communication unit 106 to the control terminal 300 contained in the record extracted from the apparatus table T20. This instruction contains the apparatus ID of the control target apparatus, the control object, the address/port, the control command, the operating condition of the command and the parameter for the command. The contents contained in the instruction are the items of information extracted from the apparatus table T20, the apparatus associative table T30 and the apparatus control table T40.

The processing unit 302 of the control terminal 300 receives the instruction of controlling the apparatus becoming the factor of disturbance via the communication unit 306. The processing unit 302 stores the received instruction in the storage unit 304. The processing unit 302 instructs the control unit 308 to acquire a present status of the apparatus contained in the received instruction. The control unit 308 acquires the present status of the apparatus contained in the instruction. The control unit 308 acquires the present status by using the control object associated with each apparatus. The control unit 308 notifies the processing unit 302 of the acquired present status of each apparatus. The processing unit 302 stores the notified present status (the status before the starting day and time) of each apparatus in the storage unit 304. The processing unit 302 instructs the control unit 308 to execute the command per apparatus contained in the received instruction. The control unit 308 performs the control based on the control command and the parameter with respect to the command by using the control object associated with each apparatus. Each apparatus performs the predetermined operation based on the control command and the parameter. Upon completing the instruction, the control unit 308 notifies the processing unit 302 that the instruction is completed. When receiving the notification of the completed instruction from the control unit 308, the processing unit 302 notifies the server 100 of the completed instruction via the communication unit 306. The processing unit 102 of the server 100 receives the notification of the completed instruction from the control terminal 300 via the communication unit 106. The processing unit 102 of the server 100 may also notify the operating terminal 400 of the completed instruction via the communication unit 106.

The processing unit 402 of the operating terminal 400 receiving the notification of the completed instruction via the communication unit 406 displays a purport that the instruction is completed (i.e., completion of the predetermined control over the equipment becoming the factor of disturbance) on the display unit 410. The user making the reservation for using the apparatus is enabled to start using the apparatus (the electron microscopic apparatus 420) by checking the display on the display unit 410. The predetermined control over the equipment becoming the factor of disturbance intends to reduce the disturbance. Hence, the user waits the notification of the completion and then starts using the apparatus (the electron microscopic apparatus 420), thereby enabling accuracy of the measurement to be improved. The control unit 408 may also perform the control to disable the electron microscopic apparatus 420 from being used as far as the processing unit 402 does not receive the notification of the completion.

The user making the reservation for using the electron microscopic apparatus 420 is enabled to use the electron microscopic apparatus 420 from the starting day and time of the reservation for the use. The electron microscopic apparatus 420 is used via the operating terminal 400. The user operates the electron microscopic apparatus 420 through the input unit 412 and other equivalent units of the operating terminal 400 and is thus enabled to check the status of the electron microscopic apparatus 420 on the display unit 410 and other equivalent units.

In S106, the processing unit 102 acquires the information of the present day and time from the timer unit 108. The processing unit 102 determines whether the present day and time is coincident with the finishing day and time of the reservation information contained in the schedule table T10 stored in the storage unit 104. When coincident with each other (S106; YES), the processing proceeds to S107. Whereas when not coincident (S106; NO), the processing diverts to S108.

In S107, the processing unit 102 of the server 100 extracts the apparatus ID of the reservation information with the finishing day and time being reached in S106. The apparatus (instanced by the electron microscopic apparatus) associated with this apparatus ID is the apparatus being employed by the user. The processing unit 102 transmits, via the communication unit 106, an instruction of getting the status of the apparatus becoming the factor of disturbance back to the status before the starting day and time to the control terminal 300 transmitting the instruction of controlling the apparatus becoming the factor of disturbance in S105 with respect to the apparatus being employed by the user.

The processing unit 302 of the control terminal 300, upon receiving the instruction from the server 100 via the communication unit 306, acquires the status of each apparatus becoming the factor of disturbance before the starting day and time, the status being stored in the storage unit 304. The processing unit 302 instructs the control unit 308 to return each apparatus becoming the factor of disturbance to the status before the starting day and time. In the same way as S105, the control unit 308 gives the instruction of returning each apparatus to the status before the starting day and time by employing the control object. Each apparatus gets, based on the instruction, the status back to the status before the starting day and time. The control unit 308, upon completing the instruction, notifies the processing unit 302 of the notification of the completed instruction. When receiving the notification of the completed instruction from the control unit 308, the processing unit 302 notifies the server 100 that the instruction is completed via the communication unit 306. The processing unit 102 of the server 100 receives the notification of the completed instruction from the control terminal 300 via the communication unit 106. The processing unit 102 of the server 100 may also notify the operating terminal 400 that the instruction is completed via the communication unit 106.

The processing unit 402 of the operating terminal 400, which receives the notification of the completed instruction via the communication unit 406, displays a purport that the instruction is completed (i.e., the completion of the process of getting the status back to the status before the starting day and time with respect to the equipment becoming the factor of disturbance) on the display unit 410. The user employing the apparatus checks the display on the display unit 410 and is thereby enabled to confirm that the status of the equipment becoming the factor of disturbance returns to the status before the starting day and time.

### Others

The server 100 may not conduct the operation in S105 till receiving an operation start signal from the operating terminal 400 that controls the reserved apparatus. This is because when the apparatus is not used even though reserved, the factor of disturbance may not be eliminated. The server 100 may not also conduct the operation in S107 till receiving an operation end signal from the operating terminal 400 that controls the reserved apparatus. This is because the return of the apparatus becoming the factor of disturbance to the status before the starting day and time becomes the factor (a decrease in measurement accuracy) of disturbance in the operation (e.g., the measurement by the electron microscopic apparatus 420). The server 100 may further conduct the operation in S107 even before reaching the finishing day and time as far as receiving the operation end signal from the operating terminal 400 that controls the reserved apparatus. This is because there is no hindrance even by returning the apparatus becoming the factor of disturbance to the status before the starting day and time as far as finishing the operation.

In S101, when displaying the reserved status screen on the display unit 208 of the reservation terminal 200, a specified usage reservation may also be cancelled. Hereat, the reservation terminal 200 notifies the server 100 of a purport that the specified usage reservation is cancelled via the communication unit 206. The processing unit 102 of the server 100, upon receiving the notification of cancelling the specified usage reservation via the communication unit 106, deletes this usage reservation from the schedule table T10 stored in the storage unit 104. The usage reservation may be thereby cancelled. The cancellation may also be made before the finishing day and time from the starting day and time onward, i.e., within the reservation time zone. In this case, the equipment is controlled in S107 as described above similarly to when reaching the finishing time in S106, and resultantly each control terminal status of the control terminal 300 is enabled to get back to the pre-using status. Note that the user allowed to execute the cancelling process is desirably limited by being identical to the user making the reservation or by authenticating a system administrator.

The day and time for controlling the apparatus becoming the factor of disturbance may also be a day and time when a time offset from the starting day and time is set. When the time offset is set, the equipment becoming the factor of disturbance may also be controlled (the operation in S104, S105) at a day and time deviated by the time offset from the starting day and time. The time offset may take a positive or negative value. The same is applied to the operation (the operation in S106, S107) related to the finishing day and time.

Any two or more apparatuses among the server 100, the reservation terminal 200, the control terminal 300 and the operating terminal 400 may integrally operate. For example, the server 100 and the reservation terminal 200 may integrally operate.

The description has been herein focused on improving the accuracy in using the electron microscopic apparatus and the ion beam radiotherapy apparatus, and it is however feasible to eliminate the disturbance when using general types of apparatuses by applying the operations described above to these general apparatuses without being limited to the electron microscopic apparatus and the ion beam radiotherapy apparatus.

### Operation and Effect of Embodiment

The system 10 according to the embodiment eliminates the factor of disturbance by controlling the equipment becoming the factor of disturbance against the measurement and other equivalent operations of the electron microscopic apparatus 420 and other equivalents in the time zone reserved for using the electron microscopic apparatus 420 and other equivalents. The factor of disturbance is eliminated when using the electron microscopic apparatus 420 and other equivalents, thereby improving the accuracy of the measurement and other equivalent operations of the electron microscopic apparatus 420 and other equivalents.

The configurations of the embodiments and examples described above may be carried out by combining these configurations to the greatest possible degree.

### Non-Transitory Computer Readable Recording Medium

A program making a computer, other machines and apparatuses (which will hereinafter be referred to as the computer and other equivalents) attain any one of the functions, can be recorded on a non-transitory recording medium readable by the computer and other equivalents. The computer and other equivalents are made to read and run the program on this non-transitory recording medium, whereby the function thereof can be provided.

Herein, the non-transitory recording medium readable by the computer and other equivalents connotes a non-transitory recording medium capable of accumulating information instanced by data, programs and other equivalents electrically, magnetically, optically, mechanically or by chemical action, which can be read from the computer and other equivalents. In such a non-transitory recording medium, computer building components instanced by the CPU and the memory are provided, and the CPU thereof may be made to run the program.

Among these non-transitory recording mediums, the mediums removable from the computer and other equivalents are exemplified by a flexible disc, a magneto-optic disc, a CD-ROM, a CD-R/W, a DVD, a DAT, an 8 mm tape, and a memory card.

A hard disc, a ROM and other equivalents are given as the non-transitory recording mediums fixed within the computer and other equivalents.

### BRIEF DESCRIPTION OF REFERENCE NUMERALS AND SYMBOLS

10 system

100 server

102 processing unit

104 storage unit

106 communication unit

108 timer unit

200 reservation terminal

202 processing unit

204 storage unit

206 communication unit

208 display unit

210 input unit

300 control terminal

302 processing unit

304 storage unit

306 communication unit

308 control unit

320 air conditioner

330 microwave oven

340 motor

400 operating terminal

402 processing unit

404 storage unit

406 communication unit

408 control unit

410 display unit

412 input unit

420 electron microscopic apparatus

90 computer

91 processor

92 memory

93 storage unit

94 input unit

95 output unit

96 communication control unit

1. A server comprising:
a storage unit to store reservation information for using a first apparatus, the reservation information containing items of information corresponding to a starting day and time and a finishing day and time for using the first apparatus; and
a processing unit to transmit a first instruction for setting a second apparatus in a predetermined status to a control terminal to control the second apparatus when reaching the starting day and time based on the reservation information stored in the storage unit, and to transmit a second instruction for setting the second apparatus in a status before the starting day and time to the control terminal when reaching the finishing day and time based on the reservation information stored in the storage unit.

2. A control system comprising:
a server; and
a control terminal,
the server including: a first storage unit to store reservation information for using a first apparatus, the reservation information containing items of information corresponding to a starting day and time and a finishing day and time for using the first apparatus; and
a first processing unit to transmit a first instruction for setting a second apparatus in a predetermined first status to the control terminal to control the second apparatus when reaching the starting day and time contained in the reservation information stored in the first storage unit, and to transmit a second instruction for setting the second apparatus in a status before the starting day and time to the control terminal when reaching the finishing day and time based on the reservation information stored in the first storage unit,
the control terminal including:
a second storage unit; and
a second processing unit,
the second processing unit acquiring a second status defined as a present status of the second apparatus from the second apparatus contained in the first instruction when receiving the first instruction from the server, storing the acquired second status in the second storage unit, then controlling based on the first instruction the second apparatus to be set in the first status, and controlling the second apparatus to be set in the second status stored in the second storage unit when receiving the second instruction from the server.

3. A control method by which a computer executes:
transmitting a first instruction for setting a second apparatus in a predetermined status to a control terminal to control the second apparatus when reaching a starting day and time based on reservation information for using a first apparatus and containing items of information corresponding to the starting day and time and a finishing day and time for using the first apparatus; and
transmitting a second instruction for setting the second apparatus in a status before the starting day and time to the control terminal when reaching the finishing day and time based on the reservation information.

4. A non-transitory computer readable medium storing a control program for making a computer execute:
transmitting a first instruction for setting a second apparatus in a predetermined status to a control terminal to control the second apparatus when reaching a starting day and time based on reservation information for using a first apparatus and containing items of information corresponding to the starting day and time and a finishing day and time for using the first apparatus; and
transmitting a second instruction for setting the second apparatus in a status before the starting day and time to the control terminal when reaching the finishing day and time based on the reservation information.
